(19) United States
(12) Patent Application Publication
(10) Pub. No.: US 2024/0066188 A1
(43) Pub. Date: Feb. 29, 2024

(54) UMBILICAL CORD BIOLOGIC STENT OR CONDUIT

(71) Applicant: Vivex Biologics Group, Inc., Atlanta, GA (US)

(72) Inventors: Ian Hodgdon, New Orleans, LA (US); Timothy Ganey, Tampa, FL (US); Adam D. McQueen, Dallas, GA (US)

(21) Appl. No.: 17/895,276

(22) Filed: Aug. 25, 2022

(51) Int. Cl. A61L 31/00; A61F 2/82; A61L 31/10

(52) U.S. Cl. CPC A61L 31/005; A61F 2/82; A61L 31/10; A61F 2002/044; A61F 2210/0076; A61F 2220/0008; A61F 2240/001; A61F 2250/0097

(57) ABSTRACT

A biologic stent or conduit made from an umbilical cord has a tissue body structure having a fluid passageway configured to be open to pass bodily fluids from a first end through a second end. The tissue body structure has an internal surface defining a boundary interior wall of the open fluid passageway and an external surface defining an exterior wall of the tissue body structure. The tissue body structure is made semi-rigid or rigid to maintain the stent or conduit open during the implantation and securing of the tissue body structure into the vessel, duct, or bowel. After a predetermined time sufficient to suture or otherwise fix the ends of the stent or conduit, the tissue body structure softens to a conformable stent inside the vessel duct, or bowel being repaired or reinforced.

### TECHNICAL FIELD

The present invention relates to a rigid or semi-rigid biologic stent or conduit made from umbilical cord having an open fluid passageway that remains open during implantation and fixing in a vessel, or duct, or bowel and thereafter softens and becomes pliable and conformable in the vessel, duct or bowel until resorbed or integrated into healing tissue without losing the functional asset of conduit during the process.

### BACKGROUND OF THE INVENTION

The use of stents in blood vessels and other fluid or solid passing tissues such as intestines, bowels or esophagus ducts is well known. In some cases, fabrication of the stents are spiral wound wires made of synthetic plastics or implantable metal wire. In other cases, the stent can be made of a biological tissue. In all cases, the stent provides an open conduit to ensure the passage of the fluid or solids remains unobstructed.

In many cases, the stent is used to repair a leakage or reinforce a bulge or weakened wall of the vessel or duct or bowel. In all cases, the use of such surgical repairs must be done with precision and great care as leakages or poor placement can result in death and even if caught in time requires a second surgery to fix which also can lead to the risk of additional damage or an increased possibility of infection.

It is therefore an object of the present invention to provide an improved stent or conduit that is more reliably implantable with greater ease and with less risk of leakage. The present invention as described herein provides a stent or conduit with an open fluid passageway made of mammalian umbilical cord that is made rigid or semi-rigid that can be fixed to a cut vessel, or duct, or bowel and transitions to a soft conformable stent or conduit after implantation as described herein.

### SUMMARY OF THE INVENTION

A biologic stent or conduit made from an umbilical cord has a tissue body structure having a fluid passageway configured to be open to pass bodily fluids from a first end through a second end. The tissue body structure has an internal surface defining a boundary interior wall of the open conduit and an external surface defining an exterior wall of the tissue body structure. The external surface is configured to connect or reinforce an inner wall of a cut vessel or duct or bowel of a patient. The tissue body structure is made semi-rigid or rigid to maintain the conduit open during the implantation and securing of the tissue body structure into the vessel, duct, or bowel. After a predetermined time sufficient to suture or otherwise fix the ends of the stent, the tissue body structure softens to a conformable stent inside the vessel duct, or bowel being repaired or reinforced.

The fluid passageway has an inner diameter or size formed on one or more mandrels or dilators to a desired size. The biologic stent is coated or perfused with a non-toxic liquid and dried forming the semi-rigid or rigid condition during manufacturing through a cryo-lyophilization step. The non-toxic fluid for coating the biologic stent is a polyampholyte cryoprotectant. The external surface of the tissue body structure can be pressed into or otherwise formed in a mold or expanded to fixed size with a clamshell restrictor.

The tissue body structure is formed by stretching the umbilical cord forming a seamless stent or conduit. Alternatively, the tissue body structure is formed by spirally winding strips of umbilical cord onto a sizing mandrel and applying heat and pressure to seal the edges of the strips together. The biologic stent or conduit is multi-layered or single-layered with a thickness (t) defined as the difference between the internal and external surface.

The biologic stent or conduit has a diameter in the range of 0.1 cm to 5.0 cm and has a length of 1 cm to 6 cm. Preferably, the biologic stent or conduit has a center with a visual indicator on the external surface located halfway between the ends of the biologic stent.

In one embodiment, the internal surface of the conduit fluid passageway has an embossed or otherwise formed flow enhancing directional pattern and the external surface has a marking indication showing the direction of flow for proper placement upon implantation.

The biologic stent or conduit can be sized to be implanted in a bowel or intestine to repair or reinforce the bowel or intestine. The biologic stent can be sized to repair or reinforce an esophagus of a patient.

A method of making a biologic stent made of umbilical cord has the steps of: cutting a length of umbilical cord; stretching the umbilical cord by placing the cut length of umbilical cord onto one or more mandrels to stretch the umbilical cord to form a conduit fluid passageway to a diameter sized to fit inside an inner wall of a vessel, a duct or a bowel; coating the stretched umbilical cord in a fluid to make the umbilical cord rigid or semi-rigid when dried; and drying the stretched coated umbilical cord to form the biologic stent or conduit.

The method further has the steps of forming a center indicator on an external surface of the biologic stent and embossing an internal surface of the biologic stent with a laminar flow enhancing directional pattern and marking an external surface with a flow direction indicator. The external surface similarly can be affixed with surface embodiments similar to a rasp to reduce slippage, enhance anchorage, and facilitate placement during suturing. The fluid making the stent rigid or semi rigid is a polyampholyte cryoprotectant.

A method of implanting a biologic stent or conduit into a vessel, or a duct or a bowel of a patient has the steps of: exposing and cutting the vessel, or duct, or bowel and inserting the rigid or semi-rigid biologic stent into the vessel, or duct, or bowel; and suturing ends of the biologic stent to the vessel, or duct, or bowel prior to the stent softening and losing rigidity, wherein the biologic stent has a center indicator on an external surface of the biologic stent that is centered in a cut portion of the vessel, or duct, or bowel prior to suturing ends of the biologic stent to the vessel, or duct, or bowel.

### BRIEF DESCRIPTION OF THE DRAWINGS

The invention will be described by way of example and with reference to the accompanying drawings in which:

FIG. 1 is an exemplary depiction of a biologic stent or conduit made from umbilical cord of the present invention.

FIG. 2 is an exemplary depiction of the biologic stent of FIG. 1 with a directional flow marking and a patterned mandrel inserted into the umbilical cord conduit.

FIG. 3 is an exemplary pattern on the mandrel of FIG. 2.

FIG. 4 is a depiction of an external patterning mandrel placed over the biologic stent of the present invention with a centerline marking.

FIG. 5A shows the biologic stent of the present invention partially inserted in a cut vessel.

FIG. 5B shows the biologic stent of the present invention fully inserted in a cut vessel.

FIG. 6 shows an alternative biologic stent formed by spirally wrapping the umbilical tissue.

FIG. 7 is an enlarged illustration of the fluid coating applied to exposed surfaces of the biologic stent.

FIG. 8 is a graph showing macrophages before and after polyampholyte fluid is added.

FIG. 9 is a graph showing tissue integrity with and without polyampholyte fluid.

FIG. 10 is a graph showing exosome concentration.

FIG. 11 shows a cut section of umbilical cord.

FIG. 12 shows an uncut umbilical cord.

FIG. 13 shows mandrels of different diameters with cut umbilical cord wrapped or stretched on the mandrels.

### DETAILED DESCRIPTION OF THE INVENTION

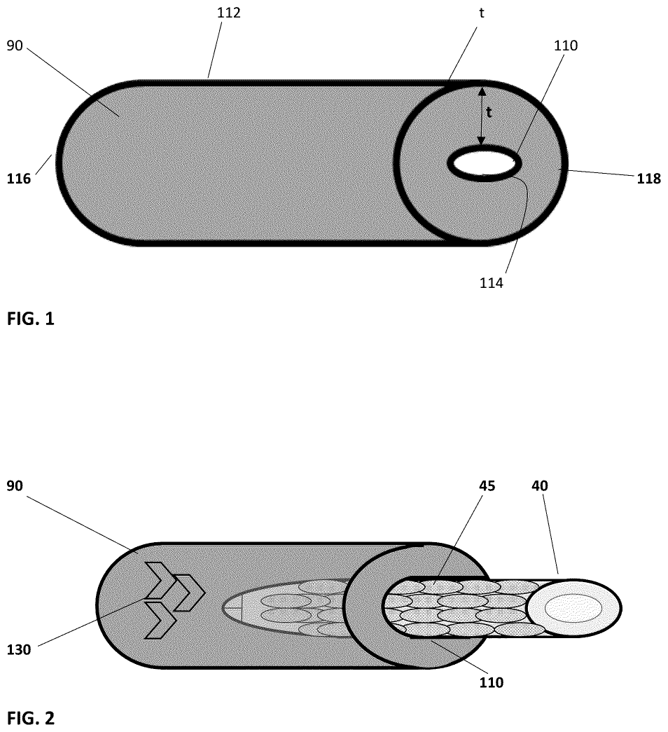

With reference to the drawings, FIGS. 1-7 show various views of the biologic stent or conduit 100 made according to the present invention. With reference to FIG. 1, a cut section of umbilical cord 90 is illustrated. The umbilical cord 90 is cut with an open lumen or fluid passageway 110. The fluid passageway 110 extends through the cut section of umbilical cord 90 from a first end 116 through to a second end 118. The cut ends 116, 118 of the naturally occurring umbilical cord tissue 90 allows fluid to pass freely through. As shown, the fluid passageway 110 is of a sufficiently small size with an exterior wall surface 112 and an interior wall surface 114 providing the wall thickness t of the present invention.

When an umbilical cord 90 is cut to a defined length it can provide biologic tissue that can be used as a stent or conduit 100. As illustrated, the wall thickness t of the umbilical cord 90 is fairly thick and therefore somewhat difficult to work with as an implant. Ideally, the wall thickness t can be reduced by taking a section of umbilical cord 90 and stretching it, the wall thickness t will decrease or thin as the length is increased. This allows for smaller diameter stents to be formed by simply cutting the stretched length of umbilical cord 90. Alternatively, some stents require a much larger diameter than the longitudinal stretched opening 110. In the cases of small vessels, the stent can be as small as a drinking straw or approximately 0.5 cm or greater with a length of approximately 4 cm to 6 cm along the longitudinal axis of the stent.

In other cases, the stent requires a much larger opening. In order to increase the diameter of the fluid passageway 110 it is ideal that the cut umbilical cord 90 be placed on a mandrel 40, the mandrel 40 has an end that is somewhat pointed or bullet shaped to allow easy insertion into the cut umbilical cord 90. As the umbilical cord 90 is pressed onto the mandrel 40, the diameter of the fluid passageway 110 is expanded as shown in FIG. 2. The diameter of the fluid passageway 110 can be expanded such that the inside diameter can exceed 4 cm making it an ideal stent for large bowels or ducts such as intestines or esophagus. When the umbilical cord 90 is expanded using mandrels 40, it achieves a stent or conduit 100 with an open fluid passageway 110 that is seamless. In other words, the umbilical cord tissue 90 itself has no seams, simply cut ends to the desired lengths as illustrated.

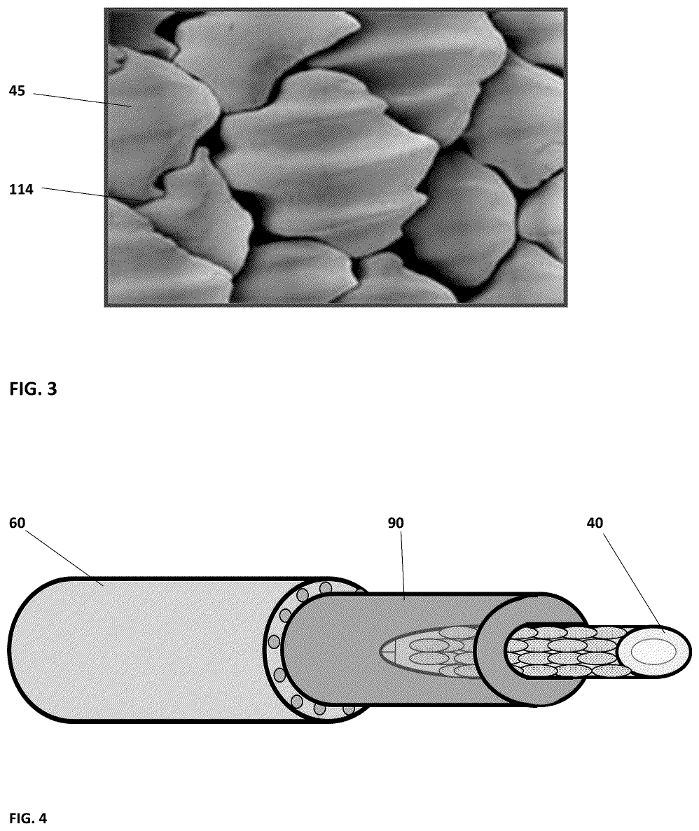

As shown in FIG. 2, the exterior surface of the mandrel 40 can have a plurality of flow enhancing patterns 45 embedded into it or embossed onto it. Accordingly, when the mandrel 40 is positioned inside the umbilical cord 90 and pressed against the interior wall 114, it provides a patterned feature as shown in FIG. 3. FIG. 3 is an enlarged view of the interior surface 114 of the fluid passageway 110 of the umbilical cord 90 once formed on a mandrel 40 with a flow enhancing pattern 45.

Ideally, when a flow enhancing pattern 45 is imparted on the interior surface 114, the exterior surface 112 of the umbilical cord 90 would be formed with an indication 130 of the preferred direction of flow, if there is one. A similar flow or traction pattern 45 could be placed on the exterior surface 112.

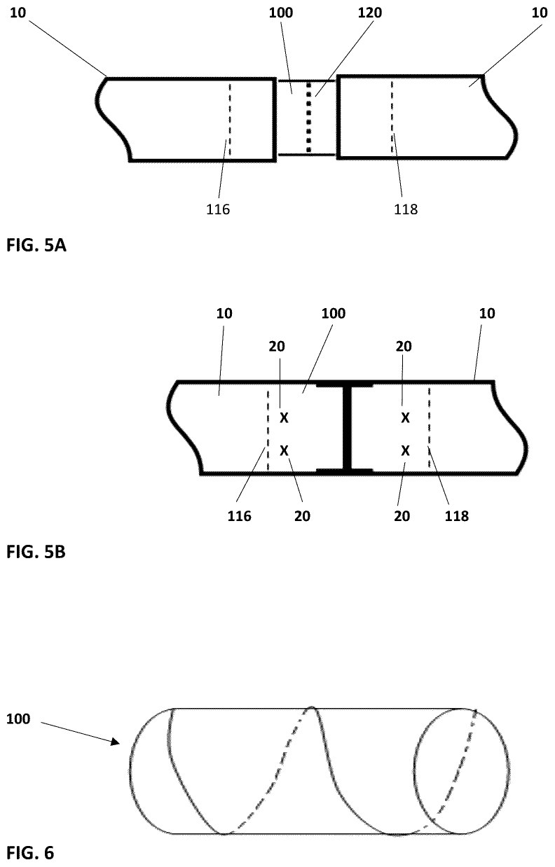

In order to do this, an exterior mandrel 60 could be applied to the cut umbilical cord 90 as it is expanded. This exterior mandrel 60 can be used to cut or emboss directional patterns 45, flow direction indicator 130, or provide a centering feature 120 as illustrated in FIG. 5A. The centering feature 120 can be embossed directly into the cut umbilical cord 90 as shown in FIG. 5A. FIG. 5A shows a cut vessel, duct or bowel 10 wherein the implant or biologic stent or conduit 100 has been partially inserted. Each end of the cut section of vessel 10 is placed over the stent or conduit 100 at each end 116, 118 of the biologic stent or conduit 100. The biologic stent or conduit 100 has a center indicator 120 as shown to ensure the biologic stent or conduit 100 is placed evenly in the cut section of vessel 10. The center indicator 120 is centered on the longitudinal length of the cut ends 116, 118 of the biologic stent or conduit 100.

Once the biologic stent or conduit 100 is positioned into the vessel, conduit or duct 10, the cut ends of the vessel, duct or bowel 10 can be pushed together so they abuttingly align on each side of the center indicator 120. At this point, the stent or conduit 100 can be sutured directly to the vessel 10. FIG. 5B shows the fully implanted biologic stent or conduit 100 centered in the cut vessel 10 and stitched or held in place with sutures or staples 20. This will cause the biologic stent or conduit 100 to be positioned properly within the vessel 10 being repaired or reinforced.

With reference to FIG. 6, an alternative way of making the biologic stent or conduit 100 of cut umbilical cord 90 is shown. The cut umbilical cord 90 is cut into strips and wound spirally to form a cylindrical biologic stent or conduit 100. When forming the stent or conduit 100, the spirally wound strips would be placed over the mandrel 40 sized with a proper diametrical opening as desired. When so formed, an exterior mandrel 60 or mold or form can be used so it can apply a pressure to the exterior surface 112 similar to FIG. 4. The mandrel and mold 40, 60 can then be heated and pressure applied to the spirally wound umbilical cord tissue 90, thus the seams forming the spiral are fused together along the edges so the cut material forms a solid walled stent or conduit 100 without any leakage. The biologic stent or conduit 100 is then formed and the thickness of the umbilical cord can be formed as so desired based on the amount of stretching of the umbilical cord tissue 90. This spiral forming of the biologic stent or conduit 100 is ideal for very large stents used in intestines or bowels. Partial sections of umbilical cord 90 could also be layered or overlapped on the mandrel 40. Alternative layering shapes as well as alternatively shaped mandrels are to alter the finished profile or dimensions of the stent are considered all within the scope of the present invention. Alternatively, due to the durability of the umbilical cord tissue 90, it can simply be stretched to size. The stent or conduit can also be shaped as a “Y”, “U”, “T”, or flared at one of both ends or can be wrapped transversely to create large sized and add additional layers if needed.

Alternatively, the stent or conduit can be cut to clean or process and then laminate, glue or adhesive, or reaffix the ends reestablishing the tube.

Regarding shaping and the epithelial asset of the perimeter of the umbilical cord having a basement membrane, in considering a direct dilation of the intact cord, the epithelial basement membrane will be in contact with the inner surface of the bowel. Alternatively, it is possible to invert the spiral design to internalize the basement membrane and allow the subepithelial connective tissue, Wharton's Jelly, to directly come into contact with inner lumen of the vessel, duct or bowel being repaired. This placement allows direct growth factor elution to the area of anastomosis that can hasten closure of the surgical repair site can possibly fill gaps or reinforce weakness. In addition, a stiffening agent such as starch can be added.

As shown, the umbilical cord tissue 90 is shown as a single layer, however, multiple layers of umbilical cord tissue can be placed over the mandrel 40 and stretched to size if so desired creating a multi-layered biologic stent or conduit 100. Regardless of the layers used, the objective is to provide a reinforcement or repair device that can be placed inside a vessel, duct or bowel to strengthen a weakened portion or repair an opening or damaged condition to prevent leakage of fluids from the vessel into the patient.

In practice, it was determined that the cut umbilical cord 90 when so manufactured provides an ideal biologic stent or conduit 100 for a such vessels. However, a significant issue of affixing the biologic stent to the vessel, duct or bowel 10 being repaired was the open lumen and the softness of the biologic tissue after being dried quickly softens due to the nature of the material of the biologic stent. Even though the biologic stent or conduit 100 is rigid when dried, it does not maintain that rigidity when exposed to body fluids or any fluids used during the procedure of implanting the biologic stent. This creates an issue of how one can staple or suture the stent into position in the vessel, duct or bowel 10. As shown, it was determined that on large vessels a minimum length of 6 cm was ideal as it allows for 3 cm on each of the stent or conduit 100 to be provided, however in order to suture the stent in place, the fluid passageway 110 must remain open for a sufficient amount of time to allow the surgeon to easily staple or suture the biologic stent or conduit 100 in place in the vessel 10 being repaired. This becomes more difficult as the stent or conduit 100 becomes soft and pliable and tends to close on itself and the vessel it is inserted into. In order to overcome this deficiency, the inventors coated the biologic stent or conduit 100 in a fluid that would allow the stent to maintain a rigid or semi rigid form during implantation. This allowed the fluid passageway 110 to remain open allowing the sutures to be easily threaded into the vessel, duct or bowel 10 and the biologic stent 110 affixing the ends into position.

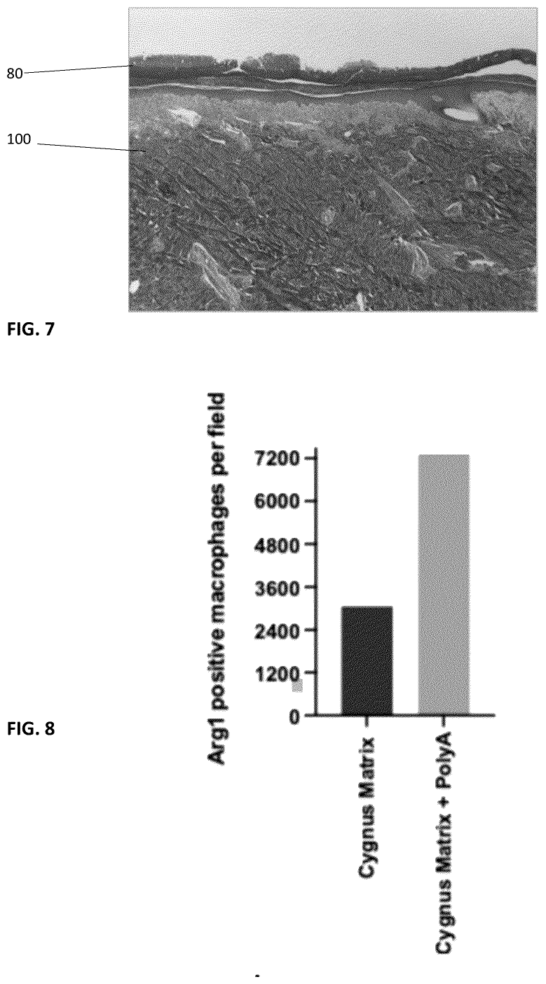

To achieve this, it was discovered a polyampholyte protectant could be applied to the dried biologic stent or conduit 100, as shown in FIG. 7. The fluid 80 is shown applied to the surface of the biologic stent or conduit 100 creating a thick layer or crust on the biologic stent or conduit 100 that allows the surgeon to suture and implant the stent or conduit 100 which remains rigid and open during the implant procedure, preferably within 30 minutes. The biologic stent or conduit 100 will soften due to exposure to bodily fluids, this occurs as the fluid coating is absorbed into the body over a period of time. Ideally, the coating is sufficiently thick to allow the stent to remain its rigidity for approximately 30 minutes before becoming flexible and pliable in the vessel, duct or bowel into which it has been implanted. This creates an ideal repair or reinforcement of the damaged vessel, duct or bowel.

The cryoprotectant is preferably a polyampholyte tissue protectant and is non-toxic. The coating provides a reduction in inflammation at the wound site and enhances healing of the wound. The coating need not and preferably is not washed away when the biologic stent is implanted in the vessel, duct or bowel. While the polyampholyte protectant is a preferred fluid for creating this rigid or semi rigid condition, it has been found that similar fluids may be used in such applications that impart a temporary rigidity only lasting a period of time prior to the coating being absorbed into the body and the biologic stent resuming a soft pliable condition after being implanted and properly sutured into the vessel, duct or bowel being repaired. Some examples of such fluids are modified amino acid cryopreservative, crystalloid, etc. cryoprotectant which might be used singularly or in conjunction with other agents. In one embodiment, the cryoprotectant or lyoprotectant is a saccharide, in particular a mono- or disaccharide. such as for example sucrose, trehalose, mannitol.

In another embodiment, the cryoprotectant or lyoprotectant is a polymer, built from substituted amino acids either singular or in substituted alterations to comport specifics of charge and topograhic charge to the polyampholyte. Still another example for consideration would be polyvinyl pyrrolidone, e.g. PVP K12, PVP K15 or PVP K17, PVP K15 and PVP K17 as examples. In another embodiment, the cryoprotectant or lyoprotectant is a mixture of a saccharide and a polymer, e.g. a mixture of Polyampholyte, PVP and trehalose. The cryoprotectant or lyoprotectant may further improve the stability of the particle size distribution during storage, including long term storage, of the freeze-dried stent and control for clumping, elution, and maintain inherent factors of living tissue.

With reference to FIGS. 8-10 various charts are shown illustrating the relative characteristics of the umbilical cord tissue vs the umbilical cord tissue treated with polyampholyte.

With reference to FIG. 8, a regenerative enhancement—macrophage trajectory graph is shown. Dramatic increase in M2a macrophage staining and cell proliferation in human amnion-chorion membrane (HACM) preserved with polyampholyte (PolyA). ImageJ software was utilized to quantitate the Arginase 1 (ARG1) positive macrophages per field.

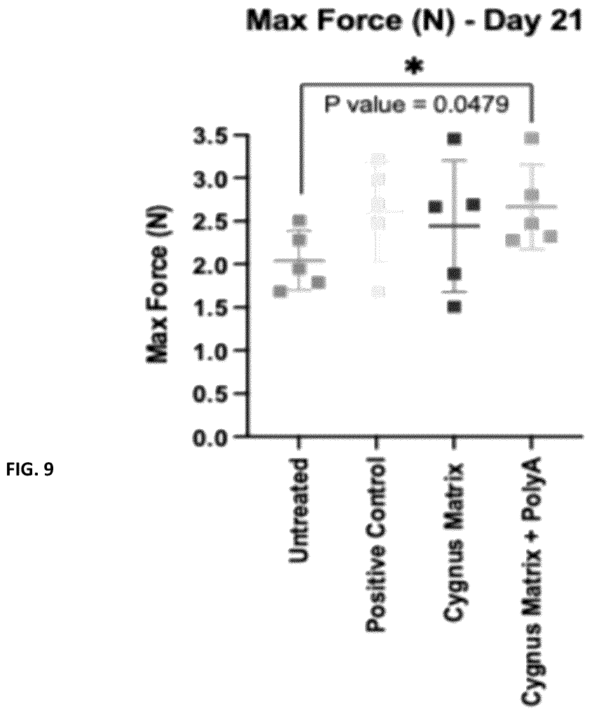

With reference to FIG. 9, a biomechanical integrity graph is shown showing the max force in Newtons on day 21. Biomechanical measurement of the tissue integrity after wound healing was measured using an ADMET® Expert 7600 material testing device. HACM preserved with PolyA has the best tissue integrity based on force break measurements. The strength of the tissue is an indication of effectiveness of tissue remodeling and wound healing.

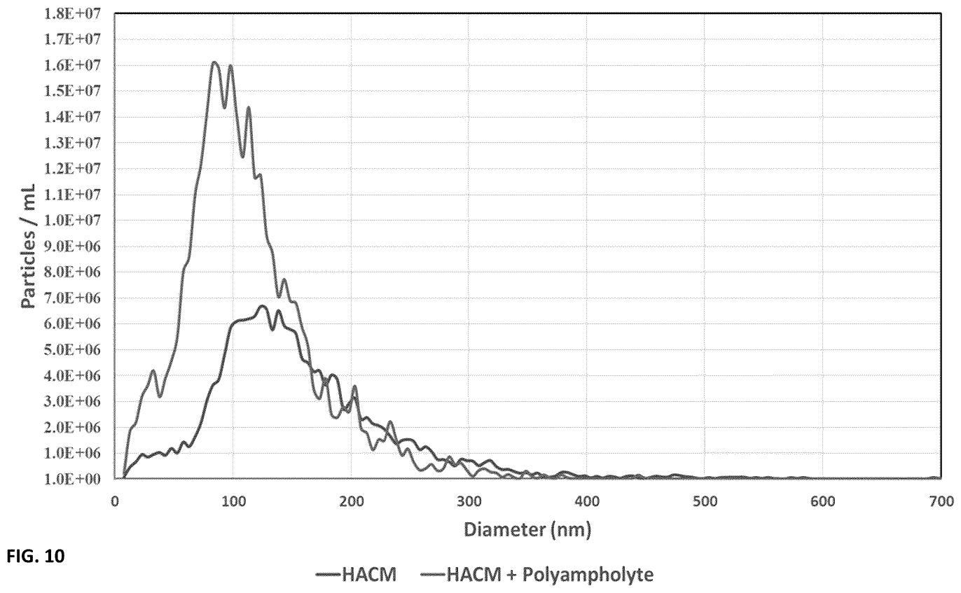

With reference to FIG. 10, an exosome/endosome isolation graph is shown. In vitro analysis of the HACM vs preserved HACM suggests more particles/ml present in the polyampholyte preserved samples than the HACM alone. For the HACM group we observed 8.710 per ml while the preserved HACM group had 1.6011 per ml (1.8× more). The preservation of an abundance of smaller diameter particles reduced the average diameter from 115 nm to 82 nm. The average diameter for the preserved HACM samples was 29% less than HACM alone.

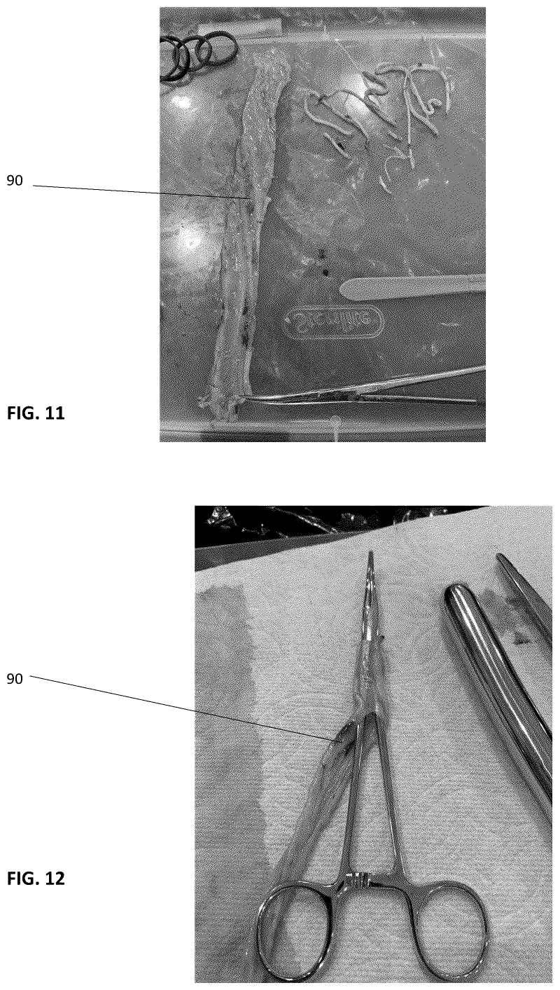

FIG. 11 shows an exemplary cut section of umbilical cord 90 prior to stretching or wrapping on a mandrel or dilator 40. Several portions of umbilical vessels are shown that have been removed from the umbilical cord 90 by simply grasping an end with a hemostat and pulling. In an exemplary procedure, after removing the vessels, the “hollowed” umbilical cord 90 is dilated on the mandrel 40 and then infused prior to expanding, and lyophilization. Vacuum infiltration is a step to assure that the cryoprotectant perfuses the umbilical cord tissue after and during the expansion and prior to the cryolyophilization/drying.

FIG. 12 shows an uncut umbilical cord 90.

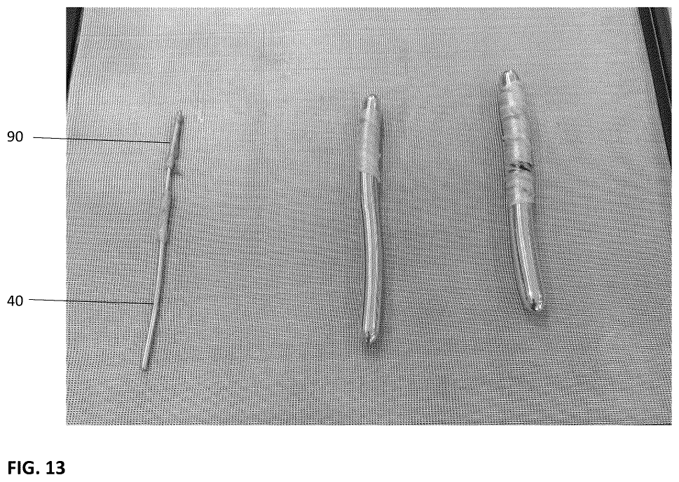

FIG. 13 shows cut sections of umbilical cord 90 wrapped or stretched on different sized mandrels 40.

The biologic stent can provide the following features: offload radial and longitudinal tension, external and internal flow of material, and supply regenerative molecules to the local tissues.

Various uses for the biologic stent of the present invention can include: Pulmonary system to decrease air leaks and fistulas (likely external so no obstruction); Trachea, Bronchus, possible lung. (Respiratory Conduit); Vascular System: Can be placed like a sleeve over arterial, venous, cardiac, and lymphatic repairs and anastomoses, including arterial-venous grafts and fistulas, to decrease bleeding, infection, aneurysms, and pseudo-aneurysms. (Vascular Conduit); Urinary system: decrease leaking form ureters, bladder, and urethra. (Urinary Conduit); Reproductive: Place over or within fallopian tubes, uterus, vagina, vas deferens, prostate, penis, and urethra. (Reproductive Conduit); Long Bone system: long bones, ribs, fingers etc. contain a medullary cavity conduit containing blood vessels and yellow bone marrow. The stent can be placed like a sleeve over the fracture repair and hardware to improve incorporation and decrease infection. (Orthopedic Conduit); Nervous System, including inner ear canal: The nervous system functions as a conduit to conduct electrical activity. Place over central cord and peripheral nerve anastomoses and repairs to increase healing and decrease neuromas. (Neurologic Conduit); Muscular/Tendon System: Muscles, and the tendons they are connected to, are created out of Tubules to conduct electoral and contraction function. (Muscular Conduit); Ocular, Dental, Teeth, Cartilage; and other vessels, ducts or bowels.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims. The surgical access window described herein encompasses the dimensions presented and any and all variations applicable to the methods and surgical technique described directly or indirectly intended with this device.

What is claimed is:

1. A biologic stent or conduit made from a mammalian umbilical cord comprises:
a tissue body structure having a fluid passageway configured to be open to pass bodily fluids from a first end through a second end, the tissue body structure having an internal surface defining a boundary interior wall of the fluid passageway and an external surface defining an exterior wall of the tissue body structure wherein the external surface is configured to connect or reinforce an inner wall of a cut vessel or duct or bowel of a patient; and
wherein the tissue body structure is made semi-rigid or rigid to maintain the fluid passageway open during the implantation and securing of the tissue body structure into the vessel, duct, or bowel, and after a predetermined time sufficient to suture or otherwise fix the ends of the stent or conduit, softens to a conformable stent or conduit inside the vessel duct, or bowel being repaired or reinforced.

2. The biologic stent or conduit of claim 1, wherein the fluid passageway has an inner diameter or size formed on one or more mandrels or dilators to achieve a desired size.

3. The biologic stent or conduit of claim 1, wherein the biologic stent is coated with a non-toxic liquid and dried forming the semi-rigid or rigid condition.

4. The biologic stent or conduit of claim 2, wherein the external surface of the tissue body structure is pressed into or otherwise formed in a mold.

5. The biologic stent or conduit of claim 2, wherein the tissue body structure is formed by stretching the umbilical cord forming a seamless stent.

6. The biologic stent or conduit of claim 2, wherein the tissue body structure is formed by spirally winding strips of umbilical cord onto a sizing mandrel and applying heat and pressure to seal the edges of the strips together.

7. The biologic stent or conduit of claim 1, wherein the biologic stent or conduit has a diameter in the range of 0.1 cm to 5.0 cm.

8. The biologic stent or conduit of claim 1, wherein the biologic stent or conduit has a length of 1 cm to 6 cm.

9. The biologic stent or conduit of claim 8, wherein the biologic stent or conduit has a center with a visual indicator on the external surface located halfway between the ends of the biologic stent or conduit.

10. The biologic stent or conduit of claim 1, wherein the internal surface of the fluid passageway has an embossed or otherwise formed flow enhancing directional pattern and the external surface has a marking indication showing the direction of flow for proper placement upon implantation.

11. The biologic stent or conduit of claim 1, wherein the external surface can be affixed with surface patterns similar to a rasp to reduce slippage, enhance anchorage, and facilitate placement during suturing.

12. The biologic stent or conduit of claim 3, wherein the non-toxic fluid for coating the biologic stent or conduit is a polyampholyte cryoprotectant.

13. The biologic stent or conduit of claim 1, wherein the biologic stent or conduit is multi-layered or single-layered with a thickness (t) defined as the difference between the internal and external surface.

14. The biologic stent or conduit of claim 1, wherein the biologic stent or conduit is sized to be implanted in a bowel or intestine to repair or reinforce the bowel or intestine.

15. The biologic stent or conduit of claim 1, wherein the biologic stent or conduit is sized to repair or reinforce an esophagus of a patient.

16. A method of making a biologic stent or conduit made of umbilical cord comprises the steps of:
cutting a length of umbilical cord;
stretching the umbilical cord by placing the cut length of umbilical cord onto one or more mandrels to stretch the umbilical cord to form a stent or conduit to a diameter sized to fit inside an inner wall of a vessel, a duct or a bowel;
coating the stretched umbilical cord in a fluid to make the umbilical cord rigid or semi-rigid when dried; and
drying the stretched coated umbilical cord to form the biologic stent or conduit.

17. The method of claim 16 further comprising;
forming a center indicator on an external surface of the biologic stent or conduit.

18. The method of claim 16 further comprising;
embossing an internal surface of the biologic stent or conduit with a flow enhancing directional pattern and marking an external surface with a flow direction indicator.

19. The method of claim 16 wherein the fluid is a polyampholyte cryoprotectant.

20. A method if implanting a biologic stent or conduit into a vessel, or a duct or a bowel of a patient comprises the steps of:
exposing and cutting the vessel, or duct, or bowel and inserting the rigid or semi-rigid biologic stent or conduit into the vessel, or duct, or bowel; and
suturing ends of the biologic stent or conduit to the vessel, or duct, or bowel prior to the stent or conduit softening and losing rigidity.

21. The method of claim 20, wherein the biologic stent or conduit has a center indicator on an external surface of the biologic stent or conduit that is centered in a cut portion of the vessel, or duct, or bowel prior to suturing ends of the biologic stent or conduit to the vessel, or duct, or bowel.
